(19) United States
(12) Patent Application Publication
(10) Pub. No.: US 2026/0171194 A1
(43) Pub. Date: Jun. 18, 2026

(54) SCREENING METHOD FOR RAW MATERIALS OF HIGH-YIELD SUSTAINABLE AVIATION FUEL

(71) Applicant: Hubei Tianji Bioenergy Co., Ltd., Xiaogan (CN)

(72) Inventors: Zhigang Tang, Xiaogan (CN); Minqiang Zhong, Xiaogan (CN); Tiancheng Deng, Xiaogan (CN); Hao Lu, Xiaogan (CN); Jinhua Deng, Xiaogan (CN); Xia Li, Xiaogan (CN); Chaohua Yi, Xiaogan (CN); Zhen Chen, Xiaogan (CN)

(21) Appl. No.: 19/401,415

(22) Filed: Nov. 26, 2025

Related U.S. Application Data
(63) Continuation-in-part of application No. 18/957,659, filed on Nov. 23, 2024, now abandoned.

(30) Foreign Application Priority Data
Dec. 13, 2023 (CN) 2023117122999

(51) Int. Cl. G16C 20/10; C10G 3/00; G01N 33/22; G01N 33/28

(52) U.S. Cl. CPC G16C 20/10; G01N 33/22; G01N 33/2835; C10G 3/60; C10G 2300/1007; C10G 2400/08

(57) ABSTRACT

Disclosed is a screening method for raw materials of high-yield sustainable aviation fuel, comprising the following steps: obtaining composition and content of fatty acid in different waste animal and vegetable oils; obtaining the theoretical limit yields of sustainable aviation fuel prepared from different fatty acids; calculating the theoretical yields of sustainable aviation fuel produced from different waste animal and vegetable oils as raw materials based on their fatty acid composition and content, as well as the theoretical limit yields of sustainable aviation fuel prepared from different fatty acids; selecting appropriate animal and vegetable oils as raw materials for producing sustainable aviation fuel based on the theoretical yields of sustainable aviation fuel. This disclosure provides guidance for enterprises to efficiently produce high-yield and high-value lipid-based bio-jet fuel.

### FIELD OF THE DISCLOSURE

The disclosure relates to the technical field of aviation fuel preparation, in particular to a screening method for raw materials of high-yield sustainable aviation fuel.

### BACKGROUND

Sustainable aviation fuel (SAF), also known as bio-jet fuel, is a new type of aviation fuel produced from raw materials such as waste animal and vegetable oils and fats (e.g., vegetable oils, animal and plant fats, waste cooking oils, etc.). Compared with traditional aviation fuels, SAF stands out for its cleanliness. Aviation fuels account for approximately 80% of the total carbon emissions in the aviation industry. Due to the need for long-distance flights with heavy loads and the need to cope with different flight environments, the aviation industry has stringent performance requirements for fuels and relies on fossil fuels, making it challenging to reduce carbon emissions. The reform and upgrading of aviation fuels have become an inevitable trend, and SAF is poised to encounter development opportunities.

In order to reduce greenhouse gas emissions and expand the raw material sources for aviation fuel, the development of bio-jet fuel production technologies has increasingly garnered attention from many countries, with oil-based bio-jet fuel process equipment technology being one of them. This technology converts vegetable oils or waste fats and oils into bio-jet fuel that can be used in aircraft through a series of complex chemical reactions and physical treatments. The emergence of this new technology not only solves the problem of global surplus in edible oils but also solves the problem of food contamination caused by waste cooking oil and hogwash oil from the catering industry. At the same time, it opens up a new path for the sustainable development of the aviation industry with renewable bioenergy.

Currently, in the production of sustainable aviation fuel (SAF), particularly in the process of manufacturing new aviation fuels using waste animal and vegetable oils and fats (such as kitchen waste oil and hogwash oil) as raw materials, the yields among different companies vary significantly, ranging from 30% to 78%, with considerable variation.

### SUMMARY

The purpose of this disclosure is to provide a screening method for raw materials of high-yield sustainable aviation fuel to solve the problems of low and fluctuating yield of sustainable aviation fuel caused by the diverse sources and complex composition of waste animal and vegetable oils and fats.

According to FIG. 1, in order to solve the above technical problems, this disclosure provides a screening method for raw materials of high-yield sustainable aviation fuel, comprising the following steps:

Step 1, obtaining composition and content of fatty acids in different waste animal and vegetable oils;

Step 2, analyzing the positions of chemical bond cleavage during the hydrodeoxygenation, cracking, and isomerization processes of different fatty acids in waste animal and vegetable oils to produce sustainable aviation fuel, and obtaining the theoretical limit yield of sustainable aviation fuel prepared from different fatty acids;

According to FIG. 2, when the fatty acids are unsaturated, the theoretical limit yield (Pᵢ) of sustainable aviation fuel prepared through the hydrogenation saturation, hydrodeoxygenation, cracking, and isomerization of unsaturated fatty acids is calculated based on the theoretical limit yield (Bᵢ) of alkanes produced from the hydrogenation saturation, and hydrodeoxygenation of unsaturated fatty acids and the theoretical limit yield (Cᵢ) of sustainable aviation fuel generated from the cracking of these alkanes; the formula for calculating the theoretical limit yield (Pᵢ) of sustainable aviation fuel produced from unsaturated fatty acids is as follows:

$${P}_{i} = {B}_{i} \times {C}_{i}$$

where Pᵢ represents the theoretical limit yield of sustainable aviation fuel produced from unsaturated fatty acids, Bᵢ represents the theoretical limit yield of alkanes produced from the hydrogenation saturation, and hydrodeoxygenation of unsaturated fatty acids, and Cᵢ represents the theoretical limit yield of sustainable aviation fuel generated from the cracking of these alkanes after hydrodeoxygenation, Cᵢ is derived from the “Cracking Position Memory Effect,” whereby the cracking product distribution is established by the original double bond locations;

According to FIG. 2, when the fatty acids are saturated, the theoretical limit yield (Bᵢ) of alkanes produced from the hydrodeoxygenation of saturated fatty acids is taken as the theoretical limit yield (Pᵢ) of sustainable aviation fuel produced from saturated fatty acids;
Step 3, based on the composition and content of fatty acids in waste animal and vegetable oils, as well as the theoretical limit yields of sustainable aviation fuel prepared from different fatty acids, calculating the theoretical yields of sustainable aviation fuel produced from different waste animal and vegetable oils as raw materials;

The formula for calculating the theoretical yield of sustainable aviation fuel is as follows:

$$η = {{\sum }^{n}}_{i = 1} ( {A}_{i} \times {P}_{i} )$$

where Aᵢ represents the content of different fatty acids in waste animal and vegetable oils, Pᵢ represents the theoretical limit yield of sustainable aviation fuel prepared from different fatty acids, and η represents the theoretical yield of sustainable aviation fuel;
Step 4, based on the theoretical yield of sustainable aviation fuel, selecting appropriate animal and vegetable oils as raw materials for producing sustainable aviation fuel.

The disclosure provides a screening method for raw materials of high-yield sustainable aviation fuel, the core of which lies in establishing a theoretical potential evaluation model based on raw material molecular structures. It should be emphasized that the purpose of the disclosure is not to precisely predict absolute yields under specific process conditions, but to calculate a unified and simplified theoretical benchmark value in order to compare the potential advantages and disadvantages of different animal and vegetable oils as raw materials, thereby providing rapid and effective priority guidance for raw material selection in industrial practice.

Furthermore, the waste animal and vegetable oil is one or more of the following: waste cooking oil, hogwash oil, garbage oil, oleic acid-based vegetable oils, linoleic acid-based vegetable oils, and medium-chain fatty acid-based vegetable oils.

Furthermore, the oleic acid-based vegetable oils include one or more of low-erucic acid rapeseed oil, high-erucic acid rapeseed oil, camellia oil, hogwash oil, palm oil, and olive oil; the linoleic acid-based vegetable oils include one or more of sunflower oil, cottonseed oil, and soybean oil; the medium-chain fatty acid-based vegetable oils include one or more of palm kernel oil and coconut oil.

Furthermore, the formula for calculating the theoretical limit yield (Bᵢ) of alkanes produced from the hydrogenation saturation, and hydrodeoxygenation of unsaturated fatty acids or the hydrodeoxygenation of saturated fatty acids is as follows:

$${{{B}_{i} = 3 \times M ’}_{i} / M ”}_{i} \times 100 \%$$

where M′i represents the molecular weight of alkanes produced by hydrogenation saturation and hydrodeoxygenation of unsaturated fatty acids or the molecular weight of alkanes produced by hydrodeoxygenation of saturated fatty acids, and M″i represents the molecular weight of triglycerides.

In the disclosure, the theoretical calculation of Bᵢ is based on the triglycerides as the reference molecule rather than fatty acid, which comprehensively considers the hydrogen consumption, water loss and carbon number change in the hydrogenation saturation, hydrodeoxygenation, cracking, and isomerization.

Furthermore, in step 2, when analyzing the positions of chemical bond cleavage during the hydrodeoxygenation, cracking, and isomerization processes of different fatty acids in waste animal and vegetable oils to produce sustainable aviation fuel, the decarboxylation and decarbonylation reactions of fatty acids are not taken into account. To simplify model and focus on the structural influence of raw materials, the disclosure intentionally disregards competing reaction pathways, such as decarboxylation and decarbonylation, which produce alkanes with one less carbon atom (Cₙ₋₁), and considers only the dominant hydrodeoxygenation pathway under typical hydrogenation treatment. Under this pathway, oxygen atoms in the fatty acid chain are removed as water, generating n-alkanes (Cₙ) with the same carbon number as the feedstock. This is based on the understanding that under specific conditions, the hydrodeoxygenation pathway can become the dominant pathway.

Furthermore, in step 2, when analyzing the positions of chemical bond cleavage during the hydrodeoxygenation, cracking, and isomerization processes of different fatty acids in waste animal and vegetable oils to produce sustainable aviation fuel, the re-polymerization of carbon dioxide, ethane, propane, and naphtha produced during the production process are not taken into account.

Furthermore, in step 2, when analyzing the positions of chemical bond cleavage during the hydrodeoxygenation, cracking, and isomerization processes of different fatty acids in waste animal and vegetable oils to produce sustainable aviation fuel, the catalytic cracking reactions of long-chain alkanes are also not taken into account.

Furthermore, the long-chain alkanes mentioned include pentadecane, heptadecane and octadecane.

Furthermore, in step S2, when analyzing the positions of chemical bond cleavage during the hydrodeoxygenation, cracking, and isomerization processes of different fatty acids in waste animal and vegetable oils to produce sustainable aviation fuel, the disclosure determines the theoretical yield of the target product (C8-C16 isoalkanes) and assumes perfect separation, and the losses in the actual separation process are not taken into account.

Furthermore, in step S2, when analyzing the positions of chemical bond cleavage during the hydrodeoxygenation, cracking, and isomerization processes of different fatty acids in waste animal and vegetable oils to produce sustainable aviation fuel, for saturated fatty acids, it is assumed that the alkanes produced by hydrodeoxygenation enter subsequent cracking/isomerization unit and their cracking behavior follows the conventional mechanism of random carbon-carbon bonds break, ultimately forming isoalkanes in the C8-C16 range.

Furthermore, in step S2, when analyzing the positions of chemical bond cleavage during the hydrodeoxygenation, cracking, and isomerization processes of different fatty acids in waste animal and vegetable oils to produce sustainable aviation fuel, unsaturated fatty acids are based on the “Cracking Position Memory Effect”. Namely, before hydrodeoxygenation, the unsaturated doubles bond are fully hydrogenated-saturated, during subsequent cleavage reactions, the chemical bond cleavage preferentially occurs at the carbon position where the original double bonds are located. This is because that the C-C bond formed at that position is still the weakest link in the entire molecular chain, both thermodynamically and kinetically.

Compared with existing technologies, the beneficial effects of this disclosure are: this disclosure, starting from the molecular structure of oils and fats, theoretically analyzes the potential positions of chemical bond cleavage during the hydrodeoxygenation, cracking, and isomerization processes of different fatty acids in oils and fats to produce alkanes (sustainable aviation fuel). It summarizes the logical relationship between the theoretical yield of sustainable aviation fuel and the fatty acid composition in oils and fats. Consequently, a screening method for raw materials of high-yield sustainable aviation fuel is provided, guiding the efficient and high-yield production of oil-based bio-jet fuel.

### BRIEF DESCRIPTION OF THE DRAWINGS

FIG. 1 is a process flowchart of the screening method for raw materials of high-yield sustainable aviation fuel provided by the disclosure;

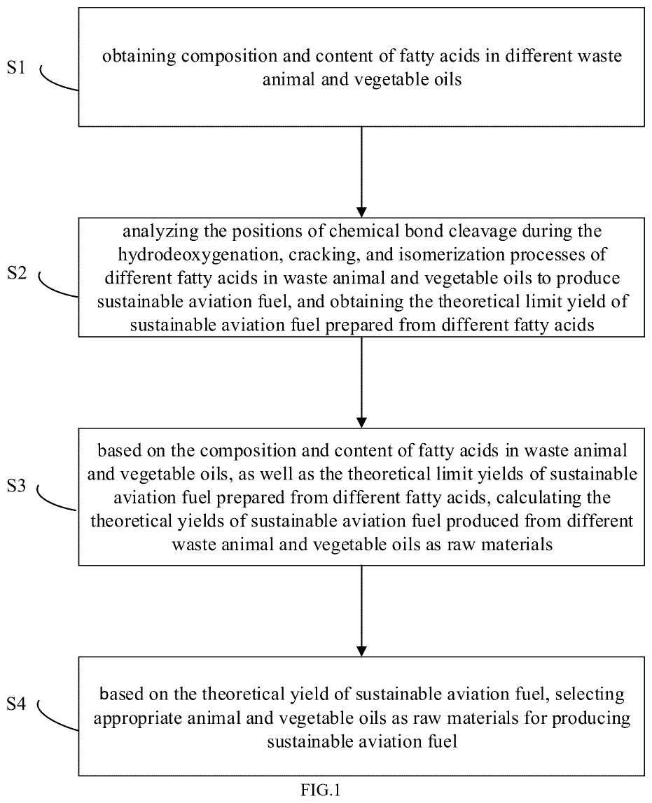

FIG. 2 is a process flowchart of obtaining the theoretical limit yield (Pᵢ) of sustainable aviation fuel prepared from different fatty acids provided by the disclosure.

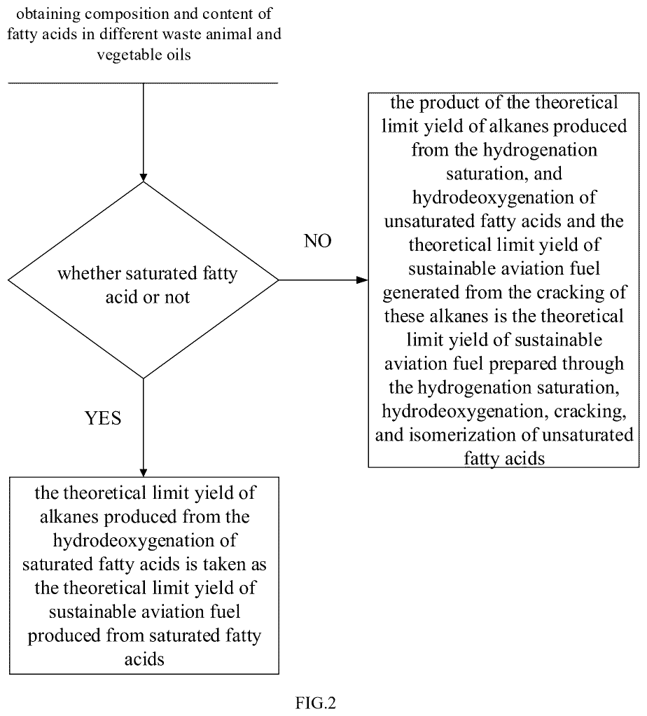

### DETAILED DESCRIPTION OF PREFERRED EMBODIMENTS

Below is a clear and comprehensive description of the technical solutions in the embodiments of this disclosure, combining with the embodiments. It is evident that the described embodiments are merely a portion of the embodiments of this disclosure, rather than exhaustive. Based on the embodiments presented in this disclosure, all other embodiments obtained by ordinary technicians in the field without creative effort fall within the scope of protection of this disclosure.

### Embodiment 1

The embodiment provides a screening method for raw materials of high-yield sustainable aviation fuel (bio-jet fuel) based on the oil composition data published in the existing data, comprising the following steps:

### (1) Obtaining Composition and Content of Fatty Acid in Different Waste Vegetable Oils;

After consulting relevant literature review, it is known that the main unsaturated fatty acid composition of European low-erucic acid rapeseed oil is approximately 72% oleic acid, 12% linoleic acid, 5% linolenic acid, and 3% erucic acid. The total unsaturated fatty acid content is approximately 92%. The saturated fatty acid content is approximately 8%, among which palmitic acid accounts for about 6% and stearic acid accounts for about 2%.

The main unsaturated fatty acid composition of Chinese high-erucic acid rapeseed oil is approximately 13% oleic acid, 12% linoleic acid, 8% linolenic acid, 9% eicosenoic acid, and 51% erucic acid. The total unsaturated fatty acid content is approximately 93%. The saturated fatty acid content is approximately 7%, among which palmitic acid accounts for about 5% and stearic acid accounts for about 2%.

The main unsaturated fatty acid composition of hogwash oil is approximately 36% oleic acid, 29% linoleic acid, and 3% linolenic acid. The total unsaturated fatty acid content is around 71%. The saturated fatty acid content is approximately 29%, among which palmitic acid accounts for about 22% and stearic acid accounts for about 7%.

The major unsaturated fatty acid composition of soybean oil is as follows: approximately 28% oleic acid, about 53% linoleic acid, and roughly 5% linolenic acid. The total unsaturated fatty acid content is approximately 86%. The saturated fatty acid content is about 14%, among which palmitic acid accounts for approximately 10% and stearic acid accounts for about 4%.

The major unsaturated fatty acid composition of palm oil is as follows: approximately 40% oleic acid and about 10% linoleic acid. The total unsaturated fatty acid content is approximately 50%. The saturated fatty acid content is about 50%, among which palmitic acid accounts for approximately 45% and stearic acid accounts for about 5%.

The fatty acid composition of palm kernel oil is as follows: approximately 9% caprylic acid, about 10% capric acid, roughly 50% lauric acid, about 15% myristic acid, and approximately 5% palmitic acid. The unsaturated fatty acid oleic acid accounts for about 10%.

The fatty acid composition of coconut oil is as follows: approximately 8% caprylic acid, about 6% capric acid, roughly 49% lauric acid, about 17% myristic acid, and approximately 13% palmitic acid. The unsaturated fatty acid oleic acid accounts for about 6%, and the unsaturated fatty acid linoleic acid is about 1%.

(2) Analyzing the Theoretical Limit Yield of Bio-Jet Fuel Produced Through Hydrodeoxygenation, Cracking, and Isomerization of Different Fatty Acids in Oils and Fats (without Considering Fatty Acid Decarboxylation and Decarbonylation Reactions, as Well as the Re-Polymerization and Utilization of Carbon Dioxide, Ethane, Propane, and Naphtha Generated During the Production Process. The Catalytic Cracking of Long-Chain Alkanes Such as Pentadecane (C15 Alkane), Heptadecane (C17 Alkane), and Octadecane (C18 Alkane) is Also not Taken into Account.)

Biomass oils such as fatty acid glycerides, fatty acid esters, and fatty acids can be directly converted into linear hydrocarbon compounds through hydrodeoxygenation, namely biomass deoxygenated oil. After hydrodeoxygenation, the linear alkanes undergo hydrocracking and isomerization, yielding alkanes with a high degree of isomerization within the C8 to C16 range, which can be utilized as bio-jet kerosene. When the fatty acid is unsaturated, all unsaturated double bonds must first undergo hydrogenation saturation to form saturated fatty acids.

The principle for preparing sustainable aviation fuel through hydrodeoxygenation, cracking, and isomerization of oils and fats is as follows:
First step: hydrogenation saturation treatment: When the fatty acid is unsaturated, all unsaturated double bonds must first undergo hydrogenation saturation to form saturated fatty acids. The reaction process is as follows:

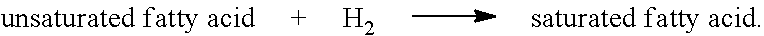

Second step: hydrodeoxygenation treatment (HDO): The saturated fatty acids undergo hydrodeoxygenation to produce linear alkanes (normal alkanes). The reaction process is as follows:

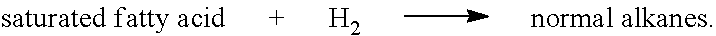

Third step: cracking and isomerization treatment: under the action of a catalyst, linear alkanes udergo cracking and hydroisomerization to obtain isoalkanes (bio-jet fuel). Based on the “Cracking Position Memory Effect”, although the double bond has been saturated, the original double bond position is still the weakest point in the carbon chain, and the cracking still tends to occur at the original double bond position.

The hydrodeoxygenation of octanoic acid produces eight-carbon alkane (octane). The theoretical limit yield of bio-jet fuel from the extreme hydrodeoxygenation of octanoic acid and octanoic acid glycerides is 72.8%.

The hydrodeoxygenation of capric acid produces ten-carbon alkane (decane). The theoretical limit yield of bio-jet fuel from the extreme hydrodeoxygenation of capric acid and capric acid glycerides is 76.93%.

The hydrodeoxygenation of lauric acid produces twelve-carbon alkane. The theoretical limit yield of bio jet fuel obtained by extreme hydrodeoxygenation of dodecanoic acid and dodecanoic acid glycerides is 79.97%.

The hydrodeoxygenation of myristic acid produces fourteen-carbon alkane. The theoretical limit yield of bio-jet fuel from the extreme hydrodeoxygenation of tetradecanoic acid and tetradecanoate glycerides is 82.30%.

The hydrodeoxygenation of palmitic acid produces sixteen-carbon alkane. The theoretical limit yield of bio-jet fuel from the extreme hydrodeoxygenation of hexadecanoic acid and hexadecanoate glycerides is 84.15%.

The hydrodeoxygenation of octadecanoic acid produces eighteen-carbon alkane. The theoretical limit yield of bio-jet fuel from the extreme hydrodeoxygenation of octadecanoic acid and octadecanoate glycerides is 85.64%.

The hydrodeoxygenation, cracking, and isomerization of oleic acid (octadecenoic acid) results in the complete cleavage of the double bond at the 9=10 position to produce two parts of nonane. The theoretical limit yield of alkanes from the extreme hydrodeoxygenation of oleic acid is 86.23%. The yield of bio-jet fuel from the catalytic cracking of the alkanes obtained through extreme hydrodeoxygenation is 100%.

The hydrodeoxygenation, cracking, and isomerization of linoleic acid (octadecadienoic acid) results in the complete cleavage of the double bonds to produce one part of nonane, one part of hexane (solvent oil), and one part of propane (liquefied petroleum gas), with the double bonds located at positions 9=10 and 12=13. The theoretical limit yield of alkanes from the extreme hydrodeoxygenation of linoleic acid is 86.82%. The yield of bio-jet fuel from the catalytic cracking of the alkanes obtained through extreme hydrodeoxygenation is 50%, with 33.3% bio-solvent oil and 16.7% liquefied petroleum gas.

The hydrodeoxygenation, cracking, and isomerization of linolenic acid (octadecatrienoic acid) results in the complete cleavage of the double bonds to produce one part of nonane and three parts of propane (liquefied petroleum gas), with the double bonds located at positions 9=10, 12=13, and 15=16. The theoretical limit yield of alkanes from the extreme hydrodeoxygenation of linolenic acid is 87.42%, the yield of bio jet fuel from the catalytic cracking of the alkanes obtained through extreme hydrodeoxygenation is 50%, with 50% liquefied petroleum gas.

The hydrodeoxygenation, cracking, and isomerization of eicosenoic acid leads to the complete cleavage of the double bond at position 11=12 to produce one part of undecane and one part of nonane. The theoretical limit yield of alkanes from the extreme hydrodeoxygenation of eicosenoic acid is 87.43%, and the yield of bio jet fuel from the catalytic cracking of the alkanes obtained through extreme hydrodeoxygenation is 100%.

The hydrodeoxygenation, cracking, and isomerization of erucic acid (docosenoic acid) leads to the complete cleavage of the double bond at position 13=14 to produce one part of tridecane and one part of nonane. The theoretical limit yield of alkanes from the extreme hydrodeoxygenation of erucic acid is 88.43%, and the yield of bio jet fuel from catalytic cracking of the alkanes through extreme hydrodeoxygenation is 100%.

The calculation process are shown in Table 1.

| | | number | | | | | |
|---|---|---|---|---|---|---|---|
| | | of | molecular | molecular | | | |
| | | carbon | weight of | weight of | | | |
| | | atoms | alkane | glyceride | Bᵢ | Cᵢ | Pᵢ |
| saturated | octanoic acid | C8 | 114.23 | 470.7 | 72.80% | | 72.80% |
| fatty acid | capric acid | C10 | 142.29 | 554.85 | 76.93% | | 76.93% |
| | dodecanoic | C12 | 170.33 | 639.00 | 79.97% | | 79.97% |
| | acid | | | | | | |
| | myristic acid | C14 | 198.39 | 723.16 | 82.30% | | 82.30% |
| | hexadecanoic | C16 | 226.44 | 807.32 | 84.15% | | 84.15% |
| | acid | | | | | | |
| | octadecanoic | C18 | 254.5 | 891.48 | 85.64% | | 85.64% |
| | acid | | | | | | |
| unsaturated | oleic acid | C18 | 254.5 | 885.43 | 86.23% | 100% | 86.23% |
| fatty acid | linoleic acid | C18 | 254.5 | 879.38 | 86.82% | 50% | 43.41% |
| | linolenic acid | C18 | 254.5 | 873.34 | 87.42% | 50% | 43.71% |
| | eicosenoic | C20 | 282.55 | 969.56 | 87.43% | 100% | 87.43% |
| | acid | | | | | | |
| | erucic acid | C22 | 310.60 | 1053.71 | 88.43% | 100% | 88.43% |

(3) Calculating the Theoretical Yield of Sustainable Aviation Fuel Prepared from Different Vegetable Oils as Raw Materials Based on the Fatty Acid Composition of these Oils and the Theoretical Limit Yield of Sustainable Aviation Fuel Obtained Through the Processes of Extreme Hydrogenation to Saturate Double Bonds, Catalytic Hydrodeoxygenation, and Catalytic Cracking of Different Fatty Acids.

### 3.1 Yield of Bio-Jet Fuel Using Hogwash Oil as Raw Material

The complete cleavage of the double bond in oleic acid (octadecenoic acid) results in a yield of nonane of 36%×86.23%×100%=31.04%, contributing to a bio-jet fuel yield of 31.04% %.

The complete cleavage of the double bonds in linoleic acid (octadecadienoic acid) results in a yield of nonane of 29%×86.82%×50%=12.59%, contributing to a bio-jet fuel yield of 12.59%.

The complete cleavage of the double bonds in linolenic acid (octadecatrienoic acid) results in a yield of nonane of 3%×87.42%×50%=1.31%, contributing to a bio-jet fuel yield of 1.31%.

The theoretical limit yield of 16-alkanes produced from the hydrodeoxygenation of hexadecanoic acid is 22%×84.15%=18.51%.

The theoretical limit yield of 18-alkanes produced from the hydrodeoxygenation of octadecanoic acid is 7%×85.64%=5.99%.

The theoretical yield of bio-jet fuel from hogwash oil is approximately 31.04%+12.59%+1.31%+18.51%+5.99%-69.45%. This means that the yield of bio-jet fuel produced from hogwash oil as a raw material is around 69.45%.

### 3.2 Yield of Bio-Jet Fuel Using Low-Erucic Acid Rapeseed Oil as Raw Material

The complete cleavage of the double bond in oleic acid (octadecenoic acid) results in a yield of nonane of 72%×86.23%×100%=62.09%, contributing to a bio-jet fuel yield of 62.09%.

The complete cleavage of the double bonds in linoleic acid (octadecadienoic acid) results in a yield of nonane of 12%×86.82%×50%=5.21%, contributing to a bio-jet fuel yield of 5.21%.

The complete cleavage of the double bonds in linolenic acid (octadecatrienoic acid) results in a yield of nonane of 5%×87.42%×50%=2.19%, contributing to a bio-jet fuel yield of 2.19%.

The complete cleavage of the double bond in erucic acid (docosenoic acid) results in a yield of tridecane and nonane of 3%×88.43%×100%=2.65%, contributing to a bio-jet fuel yield of 2.65%.

The theoretical limit yield of 16-alkanes produced from the hydrodeoxygenation of hexadecanoic acid is 6%×84.15%=5.05%.

The theoretical limit yield of 18-alkanes produced from the hydrodeoxygenation of octadecanoic acid is 2%×85.64%=1.71%.

The theoretical yield of bio-jet fuel from rapeseed oil (with low erucic acid content) is 62.09%+5.21%+2.19%+2.65%+5.05%+1.71%=78.9%. This means that the yield of bio-jet fuel produced from low-erucic acid rapeseed oil as a raw material is around 78.9%.

### 3.3 Yield of Bio-Jet Fuel Using High-Erucic Acid Rapeseed Oil as Raw Material

The complete cleavage of the double bond in oleic acid (octadecenoic acid) results in a yield of nonane of 13%×86.23%×100%=11.21%.

The complete cleavage of the double bonds in linoleic acid (octadecadienoic acid) results in a yield of nonane of 12%×86.82%×50%=5.21%.

The complete cleavage of the double bonds in linolenic acid (octadecatrienoic acid) results in a yield of nonane of 8%×87.42%×50%=3.50%.

The complete cleavage of the double bond in eicosenoic acid results in a yield of undecane and nonane of 9%×87.43%×100%=7.87%.

The complete cleavage of the double bond in erucic acid (docosenoic acid) results in a yield of tridecane and nonane of 51%×88.43%×100%=45.1%.

The theoretical limit yield of 16-alkanes produced from the hydrodeoxygenation of hexadecanoic acid is 5%×84.15%=4.21%.

The theoretical limit yield of 18-alkanes produced from the hydrodeoxygenation of octadecanoic acid is 2%×85.64%=1.71%.

The theoretical yield of bio-jet fuel from rapeseed oil (with high erucic acid content) is 11.21%+5.21%+3.50%+7.87%+45.1%+4.21%+1.71%-78.8%. This means that the yield of bio-jet fuel produced from high-erucic acid rapeseed oil as a raw material is around 78.8%.

### 3.4 Yield of Bio-Jet Fuel Using Soybean Oil as Raw Material:

The complete cleavage of the double bond in oleic acid (octadecenoic acid) results in a yield of nonane of 28%×86.23%×100%=24.14%.

The complete cleavage of the double bonds in linoleic acid (octadecadienoic acid) results in a yield of nonane of 53%×86.82%×50%=23.01%.

The complete cleavage of the double bonds in linolenic acid (octadecatrienoic acid) results in a yield of nonane of 5%×87.42%×50%=2.19%.

The theoretical limit yield of 16-alkanes produced from the hydrodeoxygenation of hexadecanoic acid is 10%×84.15%=8.415%.

The theoretical limit yield of 18-alkanes produced from the hydrodeoxygenation of octadecanoic acid is 4%×85.64%=3.43%.

The theoretical yield of bio-jet fuel from soybean oil is 24.14%+23.01%+2.19%+8.415%+3.43%=61.18%. This means that the yield of bio-jet fuel produced from soybean oil as a raw material is approximately 61.18%.

### 3.5 Yield of Bio-Jet Fuel Using Palm Oil as Raw Material:

The complete cleavage of the double bond in oleic acid (octadecenoic acid) results in a yield of nonane of 40%×86.23%×100%=34.49%.

The complete cleavage of the double bonds in linoleic acid (octadecadienoic acid) results in a yield of nonane of 10%×86.82%×50%=4.34%.

The theoretical limit yield of 16-alkanes produced from the hydrodeoxygenation of hexadecanoic acid is 45%×84.15%=37.87%.

The theoretical limit yield of 18-alkanes produced from the hydrodeoxygenation of octadecanoic acid is 5%×85.64%=4.28%.

The theoretical yield of bio-jet fuel from palm oil is 34.49%+4.34%+37.87%+4.28%=80.98%. This means that the yield of bio-jet fuel produced from palm oil as a raw material is approximately 80.98%.

### 3.6 Yield of Bio-Jet Fuel Using Palm Kernel Oil as Raw Material:

Octane yield: 9%×72.8%=6.55%;
Decane yield: 10%×76.93%=7.69%;
Dodecane yield: 50%×79.97%=39.99%;
Tetradecane yield: 15%×82.3%=12.35%;
Hexadecane yield: 5%×84.15%=4.21%;
Nonane yield: 10%×86.23%=8.623%;
The total yield of bio-jet fuel from palm kernel oil is approximately 79.41%.

### 3.7 Yield of Bio-Jet Fuel Using Coconut Oil as Raw Material:

Octane yield: 8%×72.8%=5.82%;
Decane yield: 6%×79.63%=4.62%;
Dodecane yield: 49%×79.97%=39.19%;
Tetradecane yield: 17%×82.3%=13.99%;
Hexadecane yield: 13%×84.15%=10.94%;
Nonane yield (based on oleic acid): 6%×86.23%=5.17%;
Nonane yield (based on linoleic acid): 1%×86.82%×50%=0.43%;
The total yield of bio-jet fuel from coconut oil is approximately 80.16%.

The yields of bio-jet fuel prepared from different oil sources are shown in Table 2 below.

TABLE 2

| | | | | | | Mainly Saturated | |
|---|---|---|---|---|---|---|---|
| | Mainly Unsaturated Fatty Acids | | | | | Fatty Acids | |
| | | Low-erucic | High-erucic | | | Palm | |
| Raw | Hogwash | acid | acid | Soybean | Palm | kernel | Coconut |
| Materials | oil | rapeseed oil | rapeseed oil | oil | oil | oil | oil |
| bio-jet | 69.45% | 78.9% | 78.8% | 61.18% | 80.98% | 79.41% | 80.16% |
| fuel yield | | | | | | | |

As can be seen from the table above, among the raw materials mainly composed of unsaturated fatty acids, palm oil, low-erucic acid rapeseed oil and high-erucic acid rapeseed oil have the highest theoretical yield for producing bio-jet fuel, approximately 80%. This indicates that using palm oil and rapeseed oil to produce bio-jet fuel is scientific and suitable, offers the highest yield, and generates the least amount of ineffective by-products. Therefore, to produce bio-jet fuel with a high yield, enterprises can prioritize palm oil and rapeseed oil as the raw material. For the production of bio-jet fuel from medium-chain fatty acid glycerides mainly composed of saturated fatty acids, palm kernel oil and coconut oil have comparable theoretical yields. Enterprises can select the appropriate raw material based on actual circumstances.

### Embodiment 2

The embodiment provides a screening method for raw materials of high-yield sustainable aviation fuel fuel (bio-jet fuel) based on the oil composition data used in actual production, comprising the following steps:

### (1) Obtaining Composition and Content of Fatty Acid in Different Waste Vegetable Oils;

The main unsaturated fatty acid composition of European low-erucic acid rapeseed oil is approximately 74.1% oleic acid, 10.6% linoleic acid, 4.3% linolenic acid, and 2.4% erucic acid. The total unsaturated fatty acid content is approximately 91.4%. The saturated fatty acid content is approximately 8.6%, among which palmitic acid accounts for about 5.8% and stearic acid accounts for about 2.8%.

The main unsaturated fatty acid composition of Chinese high-erucic acid rapeseed oil is approximately 11.4% oleic acid, 11.8% linoleic acid, 9.1% linolenic acid, 7.7% eicosenoic acid, and 54.2% erucic acid. The total unsaturated fatty acid content is approximately 94.2%. The saturated fatty acid content is approximately 5.8%, among which palmitic acid accounts for about 3.8% and stearic acid accounts for about 2%.

The main unsaturated fatty acid composition of hogwash oil is approximately 33.1% oleic acid, 30.5% linoleic acid, and 4.9% linolenic acid. The total unsaturated fatty acid content is around 68.5%. The saturated fatty acid content is approximately 31.5%, among which palmitic acid accounts for about 25.7% and stearic acid accounts for about 5.8%.

The major unsaturated fatty acid composition of soybean oil is as follows: approximately 30.6% oleic acid, about 49.2% linoleic acid, and roughly 6.7% linolenic acid. The total unsaturated fatty acid content is approximately 86.5%. The saturated fatty acid content is about 13.5%, among which palmitic acid accounts for approximately 8.2% and stearic acid accounts for about 5.3%.

The major unsaturated fatty acid composition of palm oil is as follows: approximately 37.5% oleic acid and about 8.7% linoleic acid. The total unsaturated fatty acid content is approximately 46.2%. The saturated fatty acid content is about 53.8%, among which palmitic acid accounts for approximately 49.2% and stearic acid accounts for about 4.6%.

The fatty acid composition of palm kernel oil is as follows: approximately 7.6% caprylic acid, about 8.9% capric acid, roughly 48.3% lauric acid, about 18.7% myristic acid, and approximately 6.7% palmitic acid. The unsaturated fatty acid oleic acid accounts for about 9.8%.

The fatty acid composition of coconut oil is as follows: approximately 8.2% caprylic acid, about 6.4% capric acid, roughly 46.1% lauric acid, about 19.3% myristic acid, and approximately 12.4% palmitic acid. The unsaturated fatty acid oleic acid accounts for about 7.3%, and the unsaturated fatty acid linoleic acid is about 0.3%.

(2) Analyzing the Theoretical Limit Yield of Bio-Jet Fuel Produced Through Hydrodeoxygenation, Cracking, and Isomerization of Different Fatty Acids in Oils and Fats. The Step is the Same as Embodiment 1.
(3) Calculating the Theoretical Yield of Sustainable Aviation Fuel Prepared from Different Vegetable Oils as Raw Materials Based on the Fatty Acid Composition of these Oils and the Theoretical Limit Yield of Sustainable Aviation Fuel Obtained Through the Processes of Extreme Hydrogenation to Saturate Double Bond, Catalytic Hydrodeoxygenation, and Catalytic Cracking of Different Fatty Acids.

The yields of bio-jet fuel prepared from different oil sources are shown in Table 3 below.

TABLE 3

| | Mainly Unsaturated Fatty Acids | | | | | Mainly Saturated | |
|---|---|---|---|---|---|---|---|
| | | Low-erucic | High-erucic | | | Fatty Acids | |
| | | acid | acid | | | Palm | |
| Raw | Hogwash | rapeseed | rapeseed | Soybean | Palm | kernel | Coconut |
| Materials | oil | oil | oil | oil | oil | oil | oil |
| bio-jet | 70.52% | 79.78% | 78.5% | 62.11% | 81.45% | 80.48% | 80.50% |
| fuel | | | | | | | |
| yield | | | | | | | |

As can be seen from the table above, the sorting order is highly consistent with embodiment 1, indicating that the method proposed in the disclosure has strong universality for the same oil with relatively stable fatty acid composition, and the sorting results have important guiding significance for selecting raw materials of high-yield sustainable aviation fuel.

(4) Determine the Optimal Practical Yield of Sustainable Aviation Fuel Prepared from Different Vegetable Oils Through Experiments.

Through systematic experimental research, the inventors determined the optimal practical process conditions for the preparation of sustainable aviation fuel from a variety of vegetable oils. The relevant experimental conditions and optimal practical yield are summarized in Table 4.

TABLE 4

| | | Low-erucic | High-erucic | | | | |
|---|---|---|---|---|---|---|---|
| | Pretreated | acid | acid | | | Palm | |
| | Hogwash | rapeseed | rapeseed | Soybean | | kernel | Coconut |
| | oil | oil | oil | oil | Palm oil | oil | oil |
| reactor | High- | fixed-bed | fixed-bed | High- | fixed-bed | fixed- | fixed- |
| | pressure | continuous | continuous | pressure | continuous | bed | bed |
| | autoclave | reactor | reactor | autoclave | reactor | continuous | continuous |
| | reactor | | | reactor | | reactor | reactor |
| catalyst | NiMo/ | CoMo/ | NiMo/ | CoMo/ | CoMo/ | Pt/SAPO- | Pt/SAPO- |
| | Al₂O₃—S | Al₂O₃—S | Al₂O₃—S | Al₂O₃—S | Al₂O₃—S | 11 | 11 |
| | (Sulfided) | (Sulfided) | (Sulfided) | (Sulfided) | (Sulfided) | | |
| temper- | 320° C. | 340° C. | 350° C. | 330° C. | 330° C. | 310° C. | 305° C. |
| ature | | | | | | | |
| H₂ | 8.0 MPa | 6.0 MPa | 7.0 MPa | 6.5 MPa | 5.5 MPa | 4.5 | 4.0 |
| pressure | | | | | | MPa | MPa |
| WHSV | 1.0 h⁻¹ | 1.5 h⁻¹ | 1.2 h⁻¹ | 1.0 h⁻¹ | 1.8 h⁻¹ | 2.0 h⁻¹ | 2.2 h⁻¹ |
| Hydrogen- | 800:1 | 1000:1 | 900:1 | 850:1 | 750:1 | 600:1 | 550:1 |
| to-oil | (v/v) | (v/v) | (v/v) | (v/v) | (v/v) | (v/v) | (v/v) |
| ratio | | | | | | | |
| Actual | 62.82% | 69.89% | 67.18% | 54.54% | 71.15% | 70.24% | 70.91% |
| yield | | | | | | | |

As can be seen from the table above, the sorting order from the industrial practices is highly consistent with that from the method provided by this disclosure. This indicates that, in order to simplify the model and highlight the influence of raw material fatty acid composition, although the disclosure focuses sonely on the main reaction pathway and does not consider the factors such as actual catalysts, temperature, and pressure, this does not impact the validity in relative raw material ranking. The experimental results of this disclosure strongly demonstrate the practical value of the described method as an effective and reliable tool for raw material pre-screening. Enterprises can make economical and efficient decisions by combining the ranking result from the method provided by this disclosure with factors such as raw material costs and supply stability.

In summary, this disclosure recognizes that current practitioners tend to approach problems from the perspective of hydrocarbon cracking in the petrochemical industry, whereas oils and fats are substances with entirely different chemical structures and distinct hydrogenation and cracking pathways compared to petroleum. This disclosure theoretically analyzes the logical relationship between the theoretical yield of sustainable aviation fuel and the fatty acid composition of oils and fats. By knowing the fatty acid composition of oils and fats, one can deduce the theoretical yield of sustainable aviation fuel produced from them. This provides guidance for enterprises to efficiently and cost-effectively produce high-yield, high-value oil-based bio-jet fuel.

It should be noted that all the embodiments mentioned above belong to the same inventive concept, and each embodiment has its own focus in description. For any aspects not fully described in a particular embodiment, reference can be made to the descriptions in other embodiments.

The embodiments described above merely represent the implementation methods of this disclosure, and the descriptions are relatively specific and detailed. However, this should not be construed as limiting the scope of this disclosure. It should be pointed out that for those skilled in the art, without departing from the concept of this disclosure, several modifications and improvements can still be made, which all fall within the protection scope of this disclosure. Therefore, the protection scope of this disclosure patent should be determined by the appended claims.

What is claimed is:

1. A screening method for raw materials of high-yield sustainable aviation fuel, comprising the following steps:
Step 1, obtaining composition and content of fatty acids in different waste animal and vegetable oils;
Step 2, analyzing the positions of chemical bond cleavage during the hydrodeoxygenation, cracking, and isomerization processes of different fatty acids in waste animal and vegetable oils to produce sustainable aviation fuel, and obtaining the theoretical limit yield of sustainable aviation fuel prepared from different fatty acids;
when the fatty acids are unsaturated, the theoretical limit yield of sustainable aviation fuel prepared through the hydrogenation saturation, hydrodeoxygenation, cracking, and isomerization of unsaturated fatty acids is calculated based on the theoretical limit yield of alkanes produced from the hydrogenation saturation, and hydrodeoxygenation of unsaturated fatty acids and the theoretical limit yield of sustainable aviation fuel generated from the cracking of these alkanes; the formula for calculating the theoretical limit yield of sustainable aviation fuel produced from unsaturated fatty acids is as follows:
$${P}_{i} = {B}_{i} \times {C}_{i}$$
where Pᵢ represents the theoretical limit yield of sustainable aviation fuel produced from unsaturated fatty acids, Bᵢ represents the theoretical limit yield of alkanes produced from the hydrogenation saturation, hydrodeoxygenation of unsaturated fatty acids, and Cᵢ represents the theoretical limit yield of sustainable aviation fuel generated from the cracking of these alkanes after hydrodeoxygenation;
when the fatty acids are saturated, the theoretical limit yield of alkanes produced from the hydrodeoxygenation of saturated fatty acids is taken as the theoretical limit yield of sustainable aviation fuel produced from saturated fatty acids;
Step 3, based on the composition and content of fatty acids in waste animal and vegetable oils, as well as the theoretical limit yields of sustainable aviation fuel prepared from different fatty acids, calculating the theoretical yields of sustainable aviation fuel produced from different waste animal and vegetable oils as raw materials;
the formula for calculating the theoretical yield of sustainable aviation fuel is as follows:
$$η = {{\sum }^{n}}_{i = 1} ( {A}_{i} \times {P}_{i} )$$
where Aᵢ represents the content of different fatty acids in waste animal and vegetable oils, Pᵢ represents the theoretical limit yield of sustainable aviation fuel prepared from different fatty acids, and n represents the theoretical yield of sustainable aviation fuel;
Step 4, based on the theoretical yield of sustainable aviation fuel, selecting appropriate animal and vegetable oils as raw materials for producing sustainable aviation fuel;
in step 2, when analyzing the positions of chemical bond cleavage during the hydrodeoxygenation, cracking, and isomerization processes of different fatty acids in waste animal and vegetable oils to produce sustainable aviation fuel, the decarboxylation and decarbonylation reactions of fatty acids are not taken into account; additionally, the re-polymerization of carbon dioxide, ethane, propane, and naphtha produced during the production process, as well as the catalytic cracking reactions of long-chain alkanes, are also not taken into account.

2. The screening method for raw materials of high-yield sustainable aviation fuel according to claim 1, in step 1, the waste animal and vegetable oil is one or more of the following: waste cooking oil, hogwash oil, garbage oil, oleic acid-based vegetable oil, linoleic acid-based vegetable oil, and medium-chain fatty acid-based vegetable oil.

3. The screening method for raw materials of high-yield sustainable aviation fuel according to claim 2, the oleic acid-based vegetable oil includes one or more of low-erucic acid rapeseed oil, high-erucic acid rapeseed oil, camellia oil, hogwash oil, palm oil, and olive oil; the linoleic acid-based vegetable oil include one or more of sunflower oil, cottonseed oil, and soybean oil; the medium-chain fatty acid-based vegetable oils include one or more of palm kernel oil and coconut oil.

4. The screening method for raw materials of high-yield sustainable aviation fuel according to claim 1, the formula for calculating the theoretical limit yield of alkanes produced from the hydrogenation saturation, hydrodeoxygenation of unsaturated fatty acids or the hydrodeoxygenation of saturated fatty acids is as follows:
$${{{B}_{i} = 3 \times M ’}_{i} / M ”}_{i} \times 100 \%$$
where M′i represents the molecular weight of alkanes produced by hydrogenation saturation and hydrodeoxygenation of unsaturated fatty acids or the molecular weight of alkanes produced by hydrodeoxygenation of saturated fatty acids, M″i represents the molecular weight of triglycerides.

5. The screening method for raw materials of high-yield sustainable aviation fuel according to claim 1, the long-chain alkanes mentioned include pentadecane, heptadecane and octadecane.
